(19) United States
(12) Patent Application Publication
(10) Pub. No.: US 2012/0040895 A1
(43) Pub. Date: Feb. 16, 2012

(54) PEPTIDE AVAILABILITY

(76) Inventors: Arie K. Kies, Pijnacker (NL); Pieter C. Pijl Van Der, Den Haag (NL)

(21) Appl. No.: 13/125,383

(22) PCT Filed: Oct. 15, 2009
(86) PCT No.: PCT/EP09/63468

§ 371 Date: Oct. 11, 2011

(30) Foreign Application Priority Data
Oct. 21, 2008 (EP) 08167097.8

(51) Int. Cl. A61K 38/00; A23L 1/212; A23K 1/00; A23J 1/00; A61P 3/02

(52) U.S. Cl. 514/5.5; 426/656; 426/618; 426/629; 426/615

(57) ABSTRACT

The present invention provides a process for producing a composition which is preferably a food, food intermediate, nutraceutical, dietary supplement, feed, or pet food, which comprises mixing together a peptide, preferably a bioactive peptide, and a dietary fibre.

The composition of the invention can be used to improve the bioavailability of bioactive peptides or used as a food, food intermediate, nutraceutical, dietary supplement, feed, or pet food.

### FIELD OF THE INVENTION

The present invention relates to the composition of a food or diet for increasing the bioavailability of specific peptides.

### BACKGROUND OF THE INVENTION

Human health may benefit from specific dietary compositions or food, called functional food. A large number of functional foods is currently available for the consumer. Margarines or milk products containing sterols to reduce blood cholesterol, fruit juices rich in antioxidant to reduce oxidative stress, and yoghurts containing bacteria (probiotics) that are claimed to improve gut health are a few examples. Also some peptides are known to benefit health condition of humans. Activities that have been described include antimicrobial and antifungal properties, blood pressure-lowering effects, cholesterol-lowering ability, antithrombotic effects, enhancement of mineral absorption, immunomodulatory effects, and localized effects on the gut (Rutherfurd-Markwick and Moughan (2005) J. AOAC Int. 88:955-966).

Peptides which are claimed to reduce blood pressure lowering include the proline-rich tripeptides Isoleucine-Proline-Proline (Ile-Pro-Pro; IPP), Valine-Proline-Poline (Val-Pro-Pro; VPP), and Leucine-Proline-Proline (Leu-Pro-Pro; LPP) (Maruyama et al. (1989) Agric. Biol. Chem. 53:1077-1081; Nakamura et al. (1995) J. Dairy Sci. 78:1253-1257). These peptides may inhibit angiotensin converting enzyme activity, an important enzyme in the rennin-angiotensin-aldosterone system, resulting in a lower blood pressure. Efficacy of bioactive peptides to lower blood pressure has been shown in different intervention trials (e.g., Hata et al. (1996) Am. J. Clin. Nutr. 64:767-771; Seppo et al. (2003) Am. J. Clin. Nutr. 77:326-330).

Because it is likely that these bioactive peptides act systemically, they need to be absorbed by the gut intact. A higher bioavailability, thus a higher part of the consumed peptides reaching the blood circulation intact, is, consequently, more efficient and will result in a higher effect or in a lower dose required to be consumed.

Bioactive peptides as IPP are normally consumed as part of a food, for instance fermented milk or a specifically hydrolyzed protein. Products containing relatively high levels of bioactive peptides may be consumed before, during, or after a meal. Bioavailability of bioactive peptides may be affected by the matrix in which it is administered to people. Possibly, oral bioavailability can be influenced by timing of consumption of the product containing these peptides, by composition of the food of which they are part of, or by composition of the meal with which it is consumed. With respect to timing of consumption of a yoghurt drink rich in IPP, VPP, or LPP, Foltz et al. ((2007) J. Nutr. 137:953-958) studied the increase of the plasma IPP concentrations after consumption of this yoghurt in overnight fasted state 30 min prior to breakfast or with breakfast. The yoghurt increased Plasma IPP level (area under the concentration vs. time curve) more than two-fold compared to a control drink, when drunk in fasted state. When consumed with the meal, the AUC increased another 30%, suggesting that combining intake of the bioactive peptide-rich yoghurt drink with a meal increased bioavailability.

It is known that food composition may affect passage rate of nutrients through the gastro-intestinal tract or their rate of absorption. For instance, gastric emptying is slower when energy density of a meal is higher (Hunt and Stubbs (1975) J. Physiol. 245:209-225). Rate of appearance of amino acids in plasma is affected by the protein composition of the meal offered (Deutz et al. (1998) J. Nutr. 128:2435-2445; Luiking et al. (2005) J. Nutr. 135:1080-1087) and by the chain length of peptides (Adibi and Morse (1977) J. Clin. Invest. 60:1008-1016; Grimble et al. (1986) Clin. Sci. 71:65-69). From the work of Deutz et al. (1998) and Luiking et al. (2005), it is learned that the plasma appearance rate of amino acids is lower when a protein of high quality is fed.

Adapting protein quality of a food may, consequently, be a method to improve bioavailability of bioactive peptides. Another method may be to prolong the residence time of digesta, containing the bioactive peptides, in the gastrointestinal tract. Residence time may be prolonged by increasing energy density of a food (e.g., Hunt et al. (1985) Gasteroenterology 89:1326-1330).

Addition of fibre to a diet may affect digesta transit time and nutrient absorption characteristics, even though results presented in the literature are not uniform (e.g., Rainbird and Low (1986) Br. J. Nutr. 55:87-98 and 111-121; Van Nieuwenhoven et al. (2001) J. Am. Coll. Nutr. 20(1):87-91). Both soluble and insoluble fibres could affect peptide absorption. Mode of action could be a reduction of transit time of digesta and thus prolonging the time in which absorption is possible. Insoluble fibres may be more of a cellulolytic-type, e.g., wheat bran, oat or barley fibre, or fibrous fractions of other cereals, pulses, or vegetables. Soluble fibres may be, e.g., pectin, guar gum, and carboxymethylcellulose.

### SUMMARY OF THE INVENTION

The invention relates to a process for producing a composition which is preferably a food, food intermediate, nutraceutical, dietary supplement, feed, or pet food, which comprises mixing a peptide, preferably a bioactive peptide, and a dietary fibre. The peptide can be a single peptide, or can be part of a peptide mixture or a peptide-containing protein hydrolysate. Preferably the peptide is a tripeptide, preferably IPP, VPP, or LPP, or the peptide mixture or peptide-containing protein hydrolysate comprises a tripeptide, preferably IPP, VPP, or LPP. The preferred fibre source is wheat bran, oat or barley fibre, (high or low methylated) pectin, guar gum, or carboxymethylcellulose.

The present invention also provides a composition which is preferably a food, food intermediate, nutraceutical, dietary supplement, feed, or pet food, which comprises a dietary fibre, which is mixed or consumed with a composition containing the bioactive peptide. In the preferred composition the bioactive peptide is a tripeptide, preferably IPP, VPP, or LPP.

Furthermore the present invention relates to a kit of parts which comprises component (a) a peptide, preferably a bioactive peptide, and component (b) a dietary fibre whereby component (a) and (b) together form a food, food intermediate, nutraceutical, dietary supplement, feed, or pet food.

The composition or the kit of parts of the invention can be used to improve the bioavailability of bioactive peptides or used as a food, food intermediate, nutraceutical, dietary supplement, feed, or pet food.

The present invention also relates to the use of a dietary fibre to improve the bioavailability of a peptide, preferably a tripeptide, more preferably IPP, VPP, or LPP.

### DETAILED DESCRIPTION OF THE INVENTION

The present invention relates to the use of food compositions that increase the bioavailability of bioactive peptides. Bioactive peptides may be administered in a variety of forms. They may be incorporated in a food, pet food, feed, dietary supplement, or a neutraceutical composition, either as pure or purified peptides, either as part of a specific protein hydrolysate. This protein hydrolysate can be produced from a large variety of proteins. These proteins can be of animal origin, e.g., milk, such as whey or casein, meat, and egg protein, of microbial origin, e.g. bacterial or yeast protein, or of vegetable origin, e.g., soy, wheat, barley, maize, bean, pea, potato, rapeseed, or linseed protein.

The present invention, therefore, relates to the use of peptides, preferably as part of a hydrolysed protein, for the preparation of a food, food intermediate, pet food, feed, dietary supplement, or neutraceutical composition, which comprises this peptide or hydrolysed protein for any bioactive property of this peptide.

The food, pet food, or feed in which the peptide, peptide mixture, or peptide-containing protein hydrolysate is mixed, or the food, pet food, or feed which is consumed in combination with the peptide, peptide mixture, or peptide-containing protein hydrolysate, contains a dietary fibre, which may either be an insoluble fibre, such as wheat bran or oat or barley fibre, or a soluble fibre, such as (high or low methylated) pectin, guar gum, or carboxymethylcellulose. The dietary fibre content of the food, pet food, feed, dietary supplement, or neutraceutical composition, is between 1 and 900 g/kg dry matter, preferably between 10 and 500 g/kg dry matter, more preferably between 15 and 200 g/kg dry matter, and most preferably between 20 an 150 g/kg dry matter. The bioactive peptide content of the food, pet food, feed, dietary supplement, or neutraceutical composition varies, depending on the active compound. For IPP, the meal as ingested contains more than 5 mg fibre/mg IPP, preferably more than 10 mg fibre/mg IPP, more preferably more than 20 mg fibre/mg IPP, even more preferably more than 30 mg fibre/mg IPP, and most preferably more than 40 mg fibre/mg IPP. For LPP, the ratio of fibre to LPP is at least 2.5 mg fibre/mg LPP, preferably more than 5 mg fibre/mg LPP, more preferably more than 7.5 mg fibre/mg LPP, even more preferably more than 10 mg fibre/mg LPP, and most preferably more than 15 mg fibre/mg LPP. For VPP, the ratio fibre to VPP is more than 5 mg fibre/mg VPP, preferably more than 50 mg fibre/mg VPP, more preferably more than 100 mg fibre/mg VPP, even more preferably more than 250 mg fibre/mg VPP, and most preferably more than 500 mg fibre/mg VPP.

Bioactive peptides may possess different beneficial properties to improve health status of the consumer. So a bioactive peptide is a peptide that may improve the health of a consumer of the peptide. For many of these effects it may be assumed that a high bioavailability is important. Typically, however, peptides are degraded to amino acids prior to absorption from the gut, or during the absorption process in the enterocytes of the gut. Measures to improve this bioavailability, e.g. by adapting the nutrient composition of the food, pet food, feed, dietary supplement, or neutraceutical composition will increase the beneficial properties of the bioactive peptide.

The present invention provides methods to increase bioavailability of bioactive peptides by co-ingesting it with a dietary fibre, which can be part of or mixed in the food, pet food, feed, dietary supplement, or neutraceutical composition in which it is comprised, or with which it is consumed.

Bioavailability, as defined herein, is oral bioavailability, i.e. the fraction of an orally administered peptide that reaches the systemic circulation. Enteral nutrition (tube feeding into stomach or intestine) is herein also considered as oral consumption. Parenteral nutrition, or suppletion of peptides by means of injection or infusion, e.g. intravenously, subcutaneously, or intraperitoneally, are herein not considered as oral administration, thus are outside the scope of current invention. Bioavailability may be expressed in absolute values, for instance as a percentage of the quantity consumed. It may also be expressed in relative values, for instance the percentual part of bioactive peptides consumed reaching the systemic circulation relative to the quantity reaching the systemic circulation under a reference or standard situation. For example, the quantity of bioactive peptide reaching the systemic circulation when consumed as part of a standard (or reference) meal is defined as 100%. The quantity of bioactive peptide reaching the systemic circulation when consumed as part of a test (or experimental) meal is expressed as a percentage relative to this standard.

Dietary fibres are the indigestible portion of (plant) foods. Generally it is accepted that fibres move food through the digestive system, absorbing water, and easing defecation. Fiber, fibre, dietary fibre, and dietary fiber have the same meaning and can be used interchangeably in the present specification.

Soluble fibre is found in varying quantities in all plant foods, including legumes (peas, soybeans, and other beans), cereals (e.g. wheat, maize or corn, oats, rye, and barley), some fruits and fruit juices (particularly apples, oranges, prune juice, plums, and berries), certain vegetables such as broccoli, carrots, and Jerusalem artichokes, root vegetables such as potatoes, sweet potatoes, and onions, and psyllium seed husk (a mucilage soluble fibre). Soluble fibres may also be fibres extracted from a vegetable source after which it is chemically or biochemically treated to modify its physical characteristics. Examples are methylation of pectin and carboxymethylation of cellulose. Legumes also typically contain shorter-chain carbohydrates indigestible by the human digestive tract but which may be metabolized by bacterial fermentation in the large intestine (colon), yielding short-chain fatty acids and gases (flatulence).

Sources of insoluble fibre include whole grain foods, bran, nuts and seeds, vegetables such as green beans, cauliflower, zucchini (courgette), and celery, the skins of some fruits, including tomatoes.

The five most fibre-rich plant foods, according to the Micronutrient Center of the Linus Pauling Institute, are legumes (15-19 grams of fibre per US cup serving, including several types of beans, lentils, and peas), wheat bran (17 grams per cup), prunes (12 grams), Asian pear (10 grams each, 3.6% by weight), and quinoa (9 grams) (source: http://en.wikipedia.org/wiki/Dietary_fiber)

A “peptide” or “oligopeptide” is defined herein as a chain of at least two amino acids that are linked through peptide bonds. The terms “peptide” and “oligopeptide” are considered synonymous (as is commonly recognized) and each term can be used interchangeably as the context requires. A “polypeptide” is defined herein as a chain containing more than 30 amino acid residues. All (oligo)peptide and polypeptide formulas or sequences herein are written from left to right in the direction from amino-terminus to carboxy-terminus, in accordance with common practice. A protein is defined as used herein as the non-hydrolyzed protein. Moreover, especially when protein is discussed in general, protein can also mean the total of polypeptides, peptides and free amino acids. A protein as used herein is defined as the non-hydrolyzed protein. The one-letter and three-letter code of amino acids used herein is commonly known in the art and can be found in Sambrook, et al. ((1989) Molecular Cloning: A Laboratory Manual, 2nd ed. Cold Spring Harbor Laboratory, Cold Spring Harbor Laboratory Press, Cold Spring Harbor, N.Y.).

Investigations have been performed to study the effect of fibre on bioavailability of peptides. In a review, Bernkop-Schnürch (2000; Int. J. Pharmaceut. 194:1-13) shows that chitosan and chitosan-derivatives may improve bioavailability of peptic pharmaceuticals. This concerns, however, the bioavailability of polypeptides such as insulin (51 amino acids; molecular weight about 5.8 kDa (Wikipedia)). Orally polypeptides have notorious low bioavailability, since they can not or badly pass the intestinal enterocytes, contradictory to free amino acids and di- and tripeptides. As Aungst et al. (1996; J. Controlled Release) write, “Peptide drugs typically have low and often unacceptable oral bioavailability, with the exceptions of some dipeptides and tripeptides.” Peptides having a size of more than ˜30 Å (Bernkop-Schnürch, 2000) or ˜500 Da (Aungst et al., 1996) are considered as too large to be absorbed in an undegraded form. Fast absorption of di- and tripeptides and free amino acids is, probably, due to the abundant availability of specialized transporters in gut enterocytes (e.g., Daniels, 2004; Annu. Rev. Physiol. 66:361-384).

Fibrous compounds would improve bioavailability of polypeptides due to enhancement of permeation of the peptides to the enterocytes, and/or to inhibition of proteases degrading the peptide (Bernkop-Schnürch, 2000). For small peptides, i.c. di- and tripeptides, such an effect cannot be expected. Intestinal proteases would degrade these peptides only to a minor extent, and permeation is already fast. Combining a peptide mixture with dietary fibre would, thus, improve bioavailability when the peptides to be delivered are large, but would have no effect when they are small (di- and tripeptides).

Probably because many pharmaceutical peptides are larger than 500 Da and a high bioavailability was expected for smaller peptides, bioavailability of such small peptides has been little investigated. Recently, however, we showed that the bioavailability of orally or intragastrically supplied synthetic tripeptides is low (Van der Pijl et al., 2008; Peptides 29:2196-2202). This is, likely, the result of extensive degradation of tripeptides in the enterocytes, rather than to low intact presentation at the enterocyte cell-wall.

That we found in our investigations an improved bioavailability of small peptides, preferably tripeptides, for example by combining a protein hydrolysate with dietary fibre, as will be shown below, is therefore a new and surprising result.

By protein hydrolysate, hydrolysate, or hydrolysed protein is meant the product that is formed by enzymatic or microbial hydrolysis of the protein. An enriched hydrolysate being a fraction of the protein hydrolysate for example enriched in selected peptides or wherein peptides or polypeptides have been removed from the hydrolysate. So an enriched hydrolysate is preferably a mixture of peptides (or a peptide mixture). The protein hydrolysate used in the present invention has a DH of between 7 and 50, preferably a DH of between 9 and 40 and most preferably between 10 and 30.

The Degree of Hydrolysis (DH) as obtained during incubation with the various proteolytic mixtures was monitored using a rapid OPA test (Nielsen et al. (2001) J. Food Sci. 66:642-646). The degree of hydrolysis is the extent to which peptide bonds are broken by the enzymatic hydrolysis reaction.

The bioactive peptide of interest may also be in a (relatively) pure form, and may be produced by means of chemical or microbiological synthesis. It may also be part of a mixture of peptides produced by such means.

Usually, foods, pet foods, and feeds, contain all macronutrients including fibre. The bioactive peptides will most often be administered in, or mixed with, a drink or yoghurt-type of food. So the composition of the invention comprising the bioactive peptide and the fibre may be consumed together, or the bioactive peptide and the fibre may be consumed separately but almost at the same time, for example during the same meal.

A dietary supplement or neutraceutical composition may or may not contain a relevant part of fibres. They may, however, be consumed prior to, during, or after a meal containing a substantial amount of fibres.

In current patent application, we will refer to fibre as part of a food, pet food, or feed in case the peptide or peptide-containing protein hydrolysate is added to this food, pet food, or feed. We refer to the fibre of a food, pet food, or feed that does not contain the bioactive peptide or peptide-containing protein hydrolysate, but which is consumed after, during, or before the intake of a dietary supplement or neutraceutical composition.

The term nutraceutical as used herein denotes the usefulness in both the nutritional and pharmaceutical field of application. Thus, novel nutraceutical compositions comprising the composition of the invention can find use as supplement to food and beverages and as pharmaceutical formulations or medicaments for enteral or parenteral application which may be solid formulations such as capsules or tablets, or liquid formulations, such as solutions, suspensions or emulsions.

Examples of Foods for Special Nutritional Uses include the categories of sport foods, slimming foods, infant formula, and clinical foods. The term dietary supplement as used herein denotes a product taken by mouth that contains a compound or mixture of compounds intended to supplement the diet. The compound or mixture of compounds in these products may include: vitamins, minerals, herbs or other botanicals, and amino acids. Dietary supplements can also be extracts or concentrates, and may be found in many forms such as tablets, capsules, softgels, gelcaps, liquids, or powders. The term nutraceutical as used herein denotes the usefulness in both the nutritional and pharmaceutical field of application. The nutraceutical compositions according to the present invention may be in any form that is suitable for administrating to the animal body including the human body, especially in any form that is conventional for oral administration, e.g. in solid form such as (additives/supplements for) food or feed, food or feed premix, tablets, pills, granules, dragées, capsules, and effervescent formulations such as powders and tablets, or in liquid form such as solutions, emulsions, or suspensions as e.g. beverages, pastes, and oily suspensions. Controlled (delayed) release formulations incorporating the hydrolysates according to the invention also form part of the invention. Furthermore, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

In general the peptide mixture or hydrolysate can be taken before, during, or after a meal. Suitable foods encompass dairy-based products, such as yoghurt, and soups or sauces. The bioactive peptide mixture or hydrolysate may also be given as a beverage. Suitable beverages encompass non-alcoholic and alcoholic drinks as well as liquid preparations to be added to drinking water and liquid food. Non-alcoholic drinks are preferably mineral water, sport drinks, fruit juices, lemonades, teas, concentrated drinks such as shots and energy drinks (for example drinks containing glucuronolactone, caffeine, and/or taurine). The bioactive peptide mixture or hydrolysate may also be incorporated in a solid food, such as a bar or candy.

As stated above food or beverage are suitably used for administration of the present invention. Beverages which can be used for the supplementation of the composition of the invention can be in the form of beverage, such as sports drinks, energy drinks, or other soft drinks, or any other suitable nutrient preparation.

A sports drink is a beverage which is supposed to rehydrate athletes, as well as restoring electrolytes, sugar, and other nutrients. Sports drinks are usually isotonic, meaning they contain the same proportions of solutes as found in the human body. (Source: http://en.wikipedia.org/wiki/Sports_drink)

Energy drinks are beverages which contain (legal) stimulants, vitamins (especially B vitamins) and minerals with the intent to give the user a burst of energy. Common ingredients include caffeine, guarana (caffeine from the Guarana plant), taurine, various forms of ginseng, maltodextrin, inositol, carnitine, creatine, glucuronolactone, and/or ginkgo biloba. They may contain high levels of sugar or glucose. Many of such beverages are flavored and/or colored. (Source: http://en.wikipedia.org/wiki/Energy_drink)

A soft drink is a drink that does not contain alcohol, as opposed to hard drinks, that do. In general, the term is used only for cold beverages. Hot chocolate, tea, and coffee are not considered soft drinks. The term originally referred exclusively to carbonated drinks, and is still commonly used in this manner. (Source: http://en.wikipedia.org/wiki/Soft_drink).

Bioavailability is the part of the consumed bioactive peptide which is absorbed intact, thus in an un-hydrolysed form, to the blood circulation. One way to measure bioavailability of a peptide is by comparing its area under the plasma-concentration time curve (AUC) with that of a reference. The reference may be the peptide after intravenous injection, in which case the absolute bioavailability is measured. In case the influence of dietary protein quality is tested, intravenous injection is no option. Thus the AUC for the bioactive peptide when administered with an investigational food is compared with its AUC after administration with a reference food, resulting in a relative bioavailability. In current patent application, foods containing fibre are compared with a food not containing fibre.

The following Examples illustrate the invention further.

### EXAMPLES

Materials and Methods. Analytical Methods in Peptide Mixtures and Protein Hydrolysates.

Amino Acid Analysis

A precisely weighed sample of the proteinous material was dissolved in dilute acid and precipitates were removed by centrifugation in an Eppendorf centrifuge. Amino acid analysis was carried out on the clear supernatant according to the PicoTag method as specified in the operators manual of the Amino Acid Analysis System of Waters (Milford Mass., USA). To that end a suitable sample was obtained from the liquid, then dried and subjected to vapour phase acid hydrolysis and derivatised using phenylisothiocyanate. The various derivatised amino acids present were quantified using HPLC methods and added up to calculate the total level of free amino acids in the weighed sample. The amino acids Cys and Trp are not included in the data obtained in this analysis.

LC/MS/MS Analysis

HPLC using an ion trap mass spectrometer (Thermoquest®, Breda, the Netherlands) coupled to a P4000 pump (Thermoquest®, Breda, the Netherlands) was used in quantification of the peptides of interest, among these the tripeptides IPP, LPP, and VPP, in the enzymatic protein hydrolysates produced. The peptides formed were separated using a Inertsil 3 ODS 3, 3 mm, 150*2.1 mm (Varian Belgium, Belgium) column in combination with a gradient of 0.1% formic acid in Milli Q water (Millipore, Bedford, Mass., USA; Solution A) and 0.1% formic acid in acetonitrile (Solution B) for elution. The gradient started at 100% of Solution A, kept here for 5 minutes, increasing linear to 5% B in 10 minutes, followed by linear increasing to 45% of solution B in 30 minutes and immediately going to the beginning conditions, and kept here 15 minutes for stabilization. The injection volume used was 50 microliter, the flow rate was 200 microliter per minute and the column temperature was maintained at 55° C. The protein concentration of the injected sample was approx. 50 microgram/milliliter.

Detailed information on the individual peptides was obtained by using dedicated MS/MS for the peptides of interest, using optimal collision energy of about 30%. Quantification of the individual peptides was performed using external calibration, by using the most abundant fragment ions observed in MS/MS mode.

The tripeptide LPP (M=325.2) was used to tune for optimal sensitivity in MS mode and for optimal fragmentation in MS/MS mode, performing constant infusion of 5 mg/ml, resulting in a protonated molecule in MS mode, and an optimal collision energy of about 30% in MS/MS mode, generating a B- and Y-ion series.

Prior to LC/MS/MS the enzymatic protein hydrolysates or bioactive peptide compositions were centrifuged at ambient temperature and 13000 rpm for 10 minutes, filtered through a 0.22 μm filter and the supernatant was diluted 1:100 with MilliQ water.

### Example 1

A study was performed using an interorgan pig model, as described by Ten Have et al. ((1996) Lab. Anim. 30:347-358). This model allows for the required intragastric (i.g.) infusions of XPP's (a peptide containing any amino acid, X, and two proline molecules; examples: IPP, LPP, and VPP) and sampling of blood from relevant veins and arteries. In this experiment, the relative bioavailabilities of three tripeptides were measured: isoleucine-proline-proline (IPP), leucine-proline-proline (LPP), and valine-proline-proline (VPP). These three peptides were chosen as an example for all bioactive peptides. They are claimed to possess blood pressure-lowering properties.

### Animals, Test Materials, and Test Procedures

Ten pathogen-free, female pigs (Dutch Landrace x Yorkshire; 8-12 weeks of age; 25.2±1.1 kg BW), provided with catheters in the stomach and abdominal aorta, were used. Methods and procedures on surgery, recovery, and animal husbandry are as described by Ten Have et al. (1996). Animals received 0.5 kg sow feed (Havens Voeders, Maashees, The Netherlands) twice daily and water was available ad libitum.
At test days, after overnight fasting, pigs received two different test meals in a randomized order. Each meal contained 32.0 g of a casein hydrolysate per 25 kg BW and a diet, which was mixed with water (˜500 ml) prior to intragastric infusion. Test meals provided 30% of the energy intake per day. Treatments varied in the composition of the diets, which were supplied by Research Diet Services, Wijk bij Duurstede, The Netherlands. The casein hydrolysate used was CasiMax, a product of the TensGuard™ family, a range of protein hydrolysates produced by DSM Food Specialties, Delft, The Netherlands. It is particularly rich in tripeptides with a C-terminal proline. IPP, LPP, and VPP concentrations in CasiMax were 5.4, 16.5, and <0.3 mg·g⁻¹ protein, respectively, at a protein content of 57%.
The following treatments were tested:
Basal (the reference diet)
Test (the test diet, a basal diet to which 21.1 g High-methylated citrus pectin was added per kg)
   Composition of the diets is presented in Table 1.
   To ensure isocaloric energy intake, quantities of the diets fed were 213.2 and 217.8 g·25 kg BW⁻¹·day⁻¹, for Basal and Test, respectively. Both test meals contained 32 g casein hydrolysate·25 kg BW⁻¹.

TABLE 1
Composition of the diets (g · kg⁻¹).

| Ingredient | Basal | Test |
|---|---|---|
| Maize starch | 295.8 | 289.5 |
| Sucrose | 147.9 | 144.8 |
| Glucose | 147.9 | 144.8 |
| Whey protein isolate | 286.4 | 280.4 |
| Soybean oil | 97.6 | 95.5 |
| Sunflower oil | 24.4 | 23.9 |
| H-M citrus pectin | 0.0 | 21.1 |

Just prior to and after administration of the meal via the gastric catheter (start was defined as t=0), blood samples were taken (at t=−5, 0, 10, 20, 40, 60, 90, 120, 150, 180, 210, 240, 270, 300, 330, and 360 min). Per time point 1 ml blood was collected in heparinized tubes and was put on crunched ice immediately.
Within one hour after collection of blood, samples were centrifuged for 5 min at 8 000 g at 4° C. Approximately 500 μl plasma was accurately weighed and transferred into a tube containing 10 μl of 100 g trifluoroacetic acid·l⁻¹ (VWR, Amsterdam, The Netherlands). After mixing, the samples were immediately frozen in liquid nitrogen and stored at −80° C. until analysis.
Samples were Analysed Using the Following Methods:

Samples of infusates were analyzed for their XPP content using the following procedure: 100 μl of the sample was mixed with 100 μl of a standard solution of universally ¹³C labelled IPP [U-¹³C-IPP] and VPP [U-¹³C-VPP] (Biopeptide Co., San Diego, Calif., USA). This mixture was vortexed for 1 min, followed by centrifugation for 20 min at 16,000 g at room temperature, after which 80 μl of the supernatant was pipetted into a 250 μl glass insert and placed into an auto sampler vial. XPP's were quantified using LC-MS (Quattro II, Micromass, Milford, Mass.).

Plasma samples were analyzed for XPP content using the following method: homogenized plasma (20 μl) was added to 50 μl internal standard solution, containing U-¹³C-IPP, U-¹³C-VPP, and U-¹³C-LPP, and 480 μl water. After mixing, this aliquot was acidified with trifluoroacetic acid to pH <3. Proteins were removed by heating the aliquot at 95° C. for 2 min, followed by centrifugation at 22,000 g for 30 min at 15° C. XPP's present in the supernatant were quantified by LC-MS (Quattro Ultima, Waters, Milford, Mass.).

### Calculations

The relative bioavailability of the Test treatment, compared to Basal, was calculated using the following equation:

$${f}_{Rel} = \frac{{AUC}_{Test} \cdot {D}_{Basal}}{{AUC}_{Basal} \cdot {D}_{Test}} \cdot 100 \%$$

where f_{Rel}=bioavailability for a given XPP from a test meal relative to Basal (%), AUC_{Test}=AUC for the test meal (nmol·l⁻¹·min⁻¹), D_{Basal}=XPP dose of the Basal meal (nmol), AUC_{Basal}=AUC for Basal (nmol·l⁻¹·min⁻¹), and D_{Test}=dose of an XPP in the test meal (nmol). AUC's were calculated from 0 to 360 min. No baseline correction was applied due to relatively low baseline XPP concentrations.

### Results

Plasma concentration time curves as observed after administration of the test meals appeared to be similar for IPP, LPP and VPP. Their relative bioavailabilities are shown in Table 2. Compared to Basal, the Test treatment resulted in higher f_{Rel}-values for IPP (P<0.10), LPP (P<0.001), and VPP (P<0.01).

TABLE 2
Relative bioavailabilities (f_{Rel}; %) for IPP, LPP,
and VPP from the Test meal as compared to Basal¹.

| | | Basal | Test |
|---|---|---|---|
| IPP | f_{Rel} | 100 | 127 ± 15 |
| LPP | f_{Rel} | 100 | 153 ± 15 |
| VPP | f_{Rel} | 100 | 141 ± 15 |

¹Values are LSmeans ± S.E.M. Basal: n = 10; Test: n = 9.

From this experiment it is evident that bioavailability of bioactive peptides can be improved by administering it with a meal containing fibre, as compared to a meal containing no fibre. In the present example high methylated citrus pectin has been used as a fibre. It is believed that also other sources of fibre, such as low-methylated citrus pectin, pectin from other sources, guar gum, carboxymethylcellulose, wheat bran, fibres from other cereals, pulses, and vegetables will give similar effects.

1. A process for producing a composition which is preferably a food, food intermediate, nutraceutical, dietary supplement, feed, or pet food, which comprises mixing together a peptide, preferably a bioactive peptide, and a dietary fibre.

2. A process according to claim 1 whereby the peptide is a single peptide, or can be part of a peptide mixture or a peptide-containing protein hydrolysate, preferably the peptide is a tripeptide, preferably IPP, VPP, or LPP or the peptide mixture or peptide-containing protein hydrolysate comprises a tripeptide, preferably IPP, VPP, or LPP.

3. A process according to claim 1 whereby the dietary fibre is an insoluble fibre, such as a by-product of the (cereal) milling industry, e.g. wheat bran or a fibre of another cereal, a fibre of pulses, e.g. of beans or peas, or of another vegetable source, or a soluble fibre, such as a pectin, e.g. high or low methylated citrus pectin, a soluble cellulose, e.g. carboxymethylcellulose, a soluble fibre extract from a plant source, e.g. guar gum.

4. A composition which is preferably a food, food intermediate, nutraceutical, dietary supplement, feed, or pet food, which comprises a mixture of a peptide, preferably a bioactive peptide, and a dietary fibre,

5. A kit of parts which comprises component (a) a peptide, preferably a bioactive peptide, and component (b) a dietary fibre whereby component (a) and (b) form together a food, food intermediate, nutraceutical, dietary supplement, feed, or pet food.

6. A composition of claim 4 whereby the peptide is a single peptide, or can be part of a peptide mixture or a peptide-containing protein hydrolysate, preferably the peptide is a tripeptide, preferably IPP, VPP, or LPP or the peptide mixture or peptide-containing protein hydrolysate comprises a tripeptide, preferably IPP, VPP, or LPP.

7. A composition of claim 4 whereby the fibre is an insoluble fibre, such as a by-product of the (cereal) milling industry, e.g. wheat bran or a fibre of another cereal, a fibre of pulses, e.g. of beans or peas, or of another vegetable source, or a soluble fibre, such as a pectin, e.g. high or low methylated citrus pectin, a soluble cellulose, e.g. carboxymethylcelluse, a soluble fibre extract from a plant source, e.g. guar gum.

8. Use of a composition according to claim 4 to improve the bioavailability of a bioactive peptide, preferably a tripeptide.

9. Use of a composition according to claim 4 as a food, food intermediate, nutraceutical, dietary supplement, feed, or pet food.

10. Use of a dietary fibre to improve the bioavailability of a peptide, preferably a tripeptide, more preferably IPP, VPP, or LPP.
